(19) United States
(12) Patent Application Publication
(10) Pub. No.: US 2004/0228818 A1
(43) Pub. Date: Nov. 18, 2004

(54) Cosmetic composition combining at least two dyes including at least one photochromic dye

(75) Inventors: Jean-Christophe Simon, Alsbach-Haemnlein (DE); Xavier Blin, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/687,645

(22) Filed: Oct. 20, 2003

Related U.S. Application Data
(60) Provisional application No. 60/434,410, filed on Dec. 19, 2002.
(60) Provisional application No. 60/434,409, filed on Dec. 19, 2002.

(30) Foreign Application Priority Data
Oct. 18, 2002 (FR) 0213038
Oct. 18, 2002 (FR) 0213036

(51) Int. Cl. A61K 7/021

(52) U.S. Cl. 424/63

(57) ABSTRACT

The present invention relates to a cosmetic composition, especially for making up the skin, the lips and/or the integuments, combining at least two dyes, including at least one photochromic dye.

### REFERENCE TO PRIOR APPLICATIONS

This application claims priority to U.S. provisional applications 60/434409 and 60/434410 both filed Dec. 19, 2002, and to French patent applications 0213038 and 0213036 both filed Oct. 18, 2002, both incorporated herein by reference.

### FIELD OF THE INVENTION

The present invention relates to a cosmetic composition, especially for making up the skin, the lips and/or the integuments, combining at least two dyes, including at least one photochromic dye.

The term “cosmetic composition” denotes a composition as defined in Council Directive 93/35/EEC of 14 Jun. 1993.

The composition according to the invention may be used especially in the field of making up or caring for keratin materials such as human skin, eyelashes, eyebrows, hair and nails. In the present case, it may be in the form of a mascara, an eyebrow product, an eyeliner, an eyeshadow, a makeup rouge, a foundation, a lip product, a body makeup product (semi-permanent tattoo) or a hair makeup product.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

One of the main functions of makeup consists in providing optical and/or color effects to the makeup areas on the skin, the lips, the eyelashes and the nails.

In general, these effects are static, i.e. they do not change either over time or as a function of the ambient luminosity. The change most frequently encountered generally consists of a gradual alteration over time of the original optical and/or color effects. This alteration is generally due either to a transfer and/or migration of the composition, these phenomena being particularly pronounced for lipsticks, or to poor behaviour of the pigments present in the makeup composition with respect to sebum, this phenomenon more particularly being encountered with foundations.

Thus, at the present time, no makeup composition exists that might be described as dynamic in terms of shade, i.e. capable of changing significantly in terms of color and/or radiance in response, for example, to a change in the ambient luminosity.

Now, it is clear that a makeup composition of this type having a dynamic optical and/or color effect would precisely satisfy a desire of consumers who are in search of novel colored makeups contrasting with conventional makeup compositions.

One main objective of the invention is, specifically, to satisfy this need.

In the present instance, the present invention especially exploits the ability manifested by certain photochromic dyes to change color when they are subjected to a radiation containing ultraviolet rays, and to do so reversibly.

The use of photochromic pigments in cosmetic compositions is known. Patent application EP 970 689 describes a two-coat makeup product for superimposing a top coat containing an ultraviolet-screening agent over a base coat containing a photochromic pigment. The corresponding makeup makes it possible to form patterns that appear or disappear depending on the nature of the light, but no dynamic effect in terms of color effect is associated therewith. Patent application WO 02/078665 proposes, for its part, cosmetic compositions comprising photochromic agents of naphthopyran type. However, these agents are incorporated into the cosmetic composition in a liquid form encapsulated in a microcapsule. This dispersible form is described as having antioxidant activity.

### DETAILED DESCRIPTION OF THE PREFERRED EMBODIMENTS

The inventors have found, unexpectedly, that it is particularly advantageous to combine at least one conventional dye with at least one specific photochromic dye to obtain a novel dynamic effect in terms of color and radiance.

More specifically, according to one of its aspects, the present invention relates to a cosmetic composition comprising, in a physiologically acceptable medium, at least two dyes, including at least one photochromic dye, characterized in that the photochromic dye has a difference in hue ΔE at least equal to 5 and is at least one naphthopyran derivative of 2H-naphtho[2,1-b]pyran type of formula (D) or 3H-naphtho[2,1-b]pyran type of formula (II) as defined below.

According to another of its aspects, the present invention is also directed towards the use of at least one photochromic dye in accordance with the invention in a cosmetic composition, especially for making up the skin, the lips and/or the integuments.

According to another of its aspects, the present invention also relates to a process for making up the skin, the lips and/or the integuments, comprising the application to the skin, the lips and/or the integuments of at least one composition in accordance with the invention.

According to a first variant, the composition according to the invention combines the photochromic dye with at least a second dye that is not photochromic in the sense of the invention, chosen preferably from goniochromatic coloring agents, water-soluble or liposoluble dyes, pigments, reflective particles and/or nacres.

According to a second variant, the cosmetic composition comprises at least two separate photochromic dyes.

The inventors have thus found that the combination of at least one photochromic dye in accordance with the invention with at least a second dye, which is different from the first and preferably more conventional and especially non-photochromic, in a cosmetic composition, especially a makeup composition, makes it possible to give this composition a rapid and reversible dynamic effect in terms of a shade and/or radiance effect.

The difference in shade is preferably sufficiently significant to be noticed with the naked eye.

It is immediate, i.e. it takes place within a very short period, either after exposing the makeup composition to UV radiation, or after stopping this exposure.

Besides this “dynamic shade and/or radiance effect” aspect, the compositions according to the invention are most particularly advantageous for compensating for the color alteration phenomenon discussed previously, which is generally associated with poor behaviour of conventional pigments with respect to sebum. Specifically, combining these pigments with a photochromic dye in accordance with the invention advantageously makes it possible to compensate for this type of alteration, since this photochromic dye is capable by itself, in response to light excitation of UV type, of yielding a colored effect which can be equivalent to that initially afforded by the pigments.

Photochromic Dye

In general, a photochromic dye is a dye that has the property of changing shade when it is illuminated with ultraviolet light and of regaining its initial color when it is no longer illuminated with this light, or of passing from an uncolored form to a colored form and vice versa. In other words, such a dye presents different shades depending on whether it is illuminated with light containing a certain amount of UV radiation, for instance sunlight, or with artificial light.

For the purposes of the present invention, the photochromic dye exhibits a difference in shade ΔE at least equal to 5.

For the purposes of the present invention, ΔE features the difference in shade observed for the photochromic dye between its excited state, i.e. in the presence of UV irradiation, and its unexcited state, i.e. in the absence of UV irradiation.

The ΔE may be determined from the chromatic space and more particularly from the specific chromatic coordinates of the photochromic dye under consideration, evaluated firstly after exposure to UV radiation for 2 minutes, generally using artificial light containing UV radiation, and secondly at rest, i.e. 5 seconds after stopping the radiation.

More specifically, the measurement protocol is as follows:

1% by weight of the photochromic dye under consideration is formulated in 100% by weight of a white lipstick base having the composition below:

| | |
|---|---|
| 2-octyldodecanol | 0.5% |
| hectorite modified with distearyl | 0.6% |
| dimethylammonium chloride | |
| liquid lanolin | 27.2% |
| microcrystalline wax | 10.5% |
| polyglycerolated (3 mol) beeswax | 4.2% |
| acetylated lanolin | 6.7% |
| arara oil (oleic acid esters) | 13.5% |
| oxypropylenated (5 PO) lanolin wax | 6.7% |
| oleyl erucate | 13.5% |
| oleic-linoleic-linolenic acid triglycerides | 1.7% |
| palmitic-oleic-linoleic acid triglycerides | 13.5% |
| sodium hyaluronate | 0.1% |
| preserving agents | 0.1% |
| vitamin | 0.5% |
| UV-screening agent | 0.7% |

A layer 50 μm thick of the composition is spread onto a Leneta brand contrast card of reference Form 1A Penopac, using an automatic spreader. The layer covers at least the white background of the card.

Reflection measurements are performed using a Minolta 3700D spectrocolorimeter. The initial trichromatic coordinates of the composition before irradiation (Lₒ, aₒ and bₒ) are thus determined. The composition is then subjected to a flux of 2 mW/cm² of a UVA radiation for two minutes, and the new trichromatic coordinates (L, a and b) are then determined immediately after stopping the irradiation. Less than 5 seconds elapse between stopping the irradiation and determining the new coordinates.

The ΔE is calculated in the following manner:

ΔE=[(L−Lₒ)²+(a−aₒ)²+(b−bₒ)²]^{1/2}

Advantageously, the photochromic dyes according to the invention have a ΔE value greater than or equal to 10, in particular greater than or equal to 25, more particularly greater than or equal to 35, or even greater than or equal to 45.

A ΔE measurement of greater than about 30 means that the photochromic dye gives a bright color.

In the context of the present invention, the photochromic dyes moreover have the advantage of responding quickly either to an excitation with UV light or to stoppage of such an excitation. This results in a rapid change in terms of coloration. This change in shade may thus be reflected in a time interval advantageously of less than or equal to 2 minutes, especially less than or equal to 1 minute and in particular less than or equal to 50 seconds.

According to one variant of the invention, the claimed composition contains at least two photochromic dyes in accordance with the invention, optionally in combination with another dye, especially a non-photochromic dye.

The photochromic dye under consideration according to the invention is generally present in an amount that is effective to give the composition a dynamic effect in terms of color and radiance. This amount may range, for example, from 0.001% to 20% by weight, especially from 0.005% to 10% by weight, in particular from 0.01% to 5% by weight and more particularly from 0.05% to 2% by weight, or even from 0.1% to 1% by weight, relative to the total weight of the cosmetic composition.

Preferably, the photochromic organic dye according to the invention is of naphthopyran type and is selected from the group consisting of 3H-naphtho[2,1-b]pyrans, which may be represented by formula (I), and from 2H-naphtho[1,2-b]pyrans, which may be represented by formula (II):

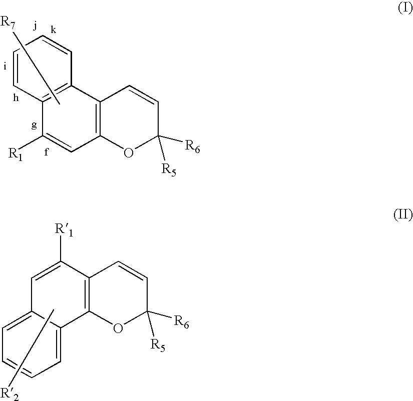

in which:

R1 represents:

(i) a hydrogen atom;

(ii) a linear, branched or cyclic, saturated or unsaturated hydrocarbon-based group containing from 1 to 30, preferably 1 to 18 and better still 1 to 12, or even 1 to 6, carbon atoms, optionally comprising 1 to 5 hetero atoms selected from the group consisting of N, O, S, Si and P, and/or optionally halogenated or perhalogenated, especially with F, Br and/or Cl;

(iii) a hydrocarbon-based ring formed with one of the bonds “f” or “gh” and the radical R₇; or

(iv) a group selected from the group consisting of —COOR₄, —C(O)NR₂R₃, —NR₂R₃, —OR₄ and —SR₄, in which:

R₂ and R₃ either represent, independently of each other, a linear, branched or cyclic, saturated or unsaturated hydrocarbon-based group containing 1 to 20, preferably 1 to 12 and better still 1 to 6 carbon atoms, optionally comprising 1 to 5 hetero atoms selected from the group consisting of N, O, S, Si and P,

or, taken together with the nitrogen atom to which they are attached, form a saturated or unsaturated hydrocarbon-based heterocycle containing 3 to 10 and preferably 4 to 6 carbon atoms and optionally 1 to 5 other hetero atoms selected from the group consisting of N, O, S, Si and P, the ring optionally being substituted with at least one linear, branched or cyclic, saturated or unsaturated hydrocarbon-based radical containing 1 to 20, preferably 1 to 15 and better still 1 to 6 carbon atoms, optionally comprising 1 to 5 hetero atoms selected from the group consisting of N, O, S, Si and P;

R₄ represents a linear, branched or cyclic, saturated or unsaturated hydrocarbon-based group containing 1 to 20, preferably 1 to 12 and better still 1 to 6 carbon atoms, which is optionally halogenated or perhalogenated especially with F, Br and/or Cl, and/or optionally comprising 1 to 5 hetero atoms selected from the group consisting of N, O, S, Si and P;

R₅ and R₆ represent, independently of each other, a group selected from the group consisting of:

(i) the saturated cyclic aminoaryl groups of formula (IIA) or (IIB):

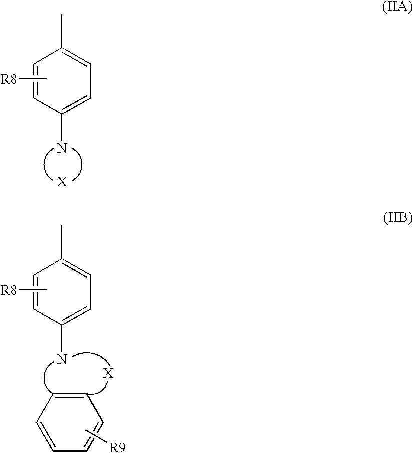

in which the ring comprising N and X is a saturated ring containing in total 3 to 30 atoms, preferably 4 to 10 and better still 5 to 8, or even 5, 6 or 7 atoms, including the nitrogen, the remainder being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20, preferably 1 to 15 and better still 1 to 6 carbon atoms, optionally comprising 1 to 5 hetero atoms selected from the group consisting of N, O, S, Si and P;

(ii) the indolinoaryl groups of formula (III):

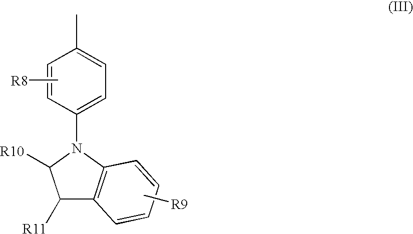

in which R₁₀ and R₁₁ represent, independently of each other, a group selected from the group consisting of (i) linear, branched or cyclic, saturated or unsaturated hydrocarbon-based groups containing 1 to 30, preferably 1 to 18 and better still 1 to 12, or even 1 to 6, carbon atoms, optionally comprising 1 to 5 hetero atoms selected from the group consisting of N, O, S, Si and P, and/or optionally halogenated or perhalogenated, especially with F, Br and/or Cl; (ii) halogen atoms, and especially F, Br and/or Cl; (iii) —CN (nitrile), —COOH (carboxylate) or —NO₂ (nitro) groups; (iv) a hydrogen atom; (v) a group selected from the group consisting of —C(O)NR₂R₃, —NR₂R₃, —OR₄ and —SR₄ with R₂, R₃ and R₄ having the meanings given above; (vi) the radicals R₁₀ and R₁₁ together possibly forming a saturated or unsaturated hydrocarbon-based ring containing in total 5 to 8 atoms (including the atoms of the indoline ring), the atoms being selected from the group consisting of C, O, S and/or NR with R representing H or a linear, branched or cyclic, saturated or unsaturated hydrocarbon-based radical containing 1 to 20 or even 1 to 12 carbon atoms, optionally comprising 1 to 5 hetero atoms selected from the group consisting of N, O, S, Si and P,

(iii) the groups of formula (IV):

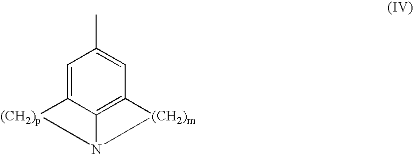

in which m and p are, independently of each other, integers ranging from 2 to 5;

(iv) the unsaturated cyclic aminoaryl groups of formula (VA), (VB) or (VC):

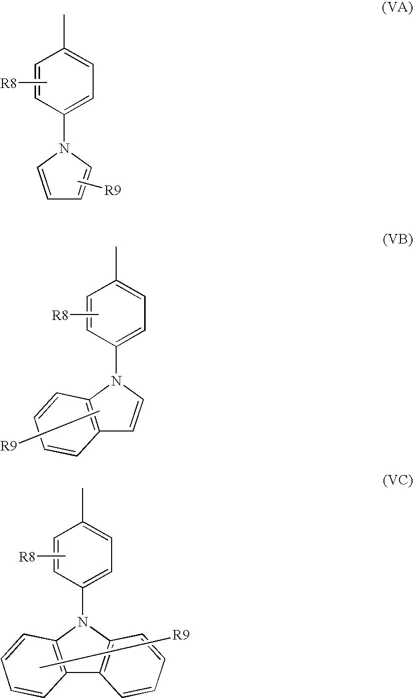

in which R₈ and R₉ represent, independently of each other, a group selected from the group consisting of (i) linear, branched or cyclic, saturated or unsaturated hydrocarbon-based groups containing 1 to 30, preferably 1 to 18 and better still 1 to 12, or even 1 to 6, carbon atoms, optionally comprising 1 to 5 hetero atoms selected from the group consisting of N, O, S, Si and P, and/or optionally halogenated or perhalogenated, especially with F, Br and/or Cl; (ii) halogen atoms, especially F, Br and/or Cl; (iii) —CN (nitrile), —COOH (carboxylate) or —NO₂ (nitro) groups; (iv) a hydrogen atom; (v) a group selected from the group consisting of —C(O)NR₂R₃, —NR₂R₃, —OR₄ and —SR₄ with R₂, R₃ and R₄ having the meanings given above;

(v) a linear, branched or cyclic, saturated or unsaturated hydrocarbon-based group containing 1 to 30, preferably 2 to 18 and better still 3 to 12 carbon atoms, optionally comprising 1 to 5 hetero atoms selected from the group consisting of N, O, S, Si and P; and especially a group selected from the group consisting of —C₆H₄—CONR₂R₃, —C₆H₄—NR₂R₃ and —C₆H₄—OR₄ with R₂, R₃ and R₄ having the meanings given above;

R₇ represents a group selected from the group consisting of:

(i) linear, branched or cyclic, saturated or unsaturated hydrocarbon-based groups containing 1 to 30, preferably 1 to 18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;

(ii) halogen atoms, and especially F, Br and/or Cl;

(iii) —CN (nitrile), —COOH (carboxylate), —NO₂ (nitro), —N═N— (azo), ═NH (imino) or —CONH₂ (amide) groups;

(iv) a hydrogen atom;

(v) a group selected from the group consisting of —C(O)NR₂R₃, —NR₂R₃, —OR₄ and —SR₄ with R₂, R₃ and R₄ having the meanings given above;

(vi) the radical R₇ also possibly forming, with one of the bonds “i”, “j”, “k” or “g,h” taken with the radical R₁, or “f” taken with the radical R₁, a saturated hydrocarbon-based ring containing in total 3 to 8, preferably 4 to 7 and better still 5 or 6 carbon atoms, optionally comprising 1 to 5 hetero atoms selected from the group consisting of N, O, S, Si and P;

R′₁ represents a group selected from the group consisting of:

a hydrogen atom;

(ii) a linear, branched or cyclic, saturated or unsaturated hydrocarbon-based group containing 1 to 30, preferably 1 to 18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;

(iii) a group selected from the group consisting of —C(O)NR₂R₃, —NR₂R₃, —OR₄ and —SR₄, with R₂, R₃ and R₄ having the meanings given above;

R′₂ represents a group selected from the group consisting of:

(i) linear, branched or cyclic, saturated or unsaturated hydrocarbon-based groups containing 1 to 30, preferably 1 to 18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;

(ii) halogen atoms, especially F, Br and/or Cl;

(iii) —CN (nitrile), —COOH (carboxylate), —NO₂ (nitro), —N═N— (azo), ═NH (imino) or —CONH₂ (amide) groups;

(iv) a hydrogen atom;

(v) a group selected from the group consisting of —C(O)NR₂R₃, —NR₂R₃, —OR₄ and —SR₄, with R₂, R₃ and R₄ having the meanings given above.

The photochromic organic dyes according to the invention may especially correspond to one of the formulae (Ia) and (IIa) below:

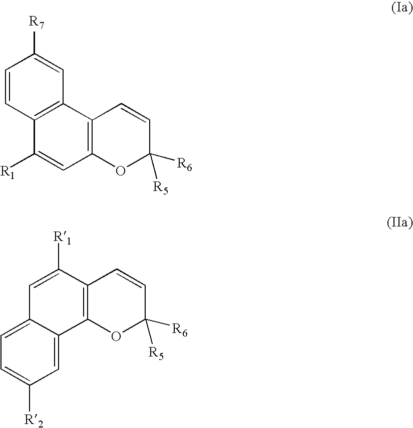

in which R₁, R₅, R₆, R₇, R′₁ and R′₂ are defined as above.

In particular, R₁ may represent a hydrogen atom; a hydrocarbon-based ring with one of the bonds “f” or “gh” and the radical R₇; or a group selected from the group consisting of —COOR₄, —NR₂R₃, —OR₄ and —SR₄, in which:

R₂ and R₃ either may represent, independently of each other, a linear, branched or cyclic, saturated or unsaturated hydrocarbon-based group containing 1 to 20, preferably 1 to 12 and better still 1 to 6 carbon atoms, optionally comprising 1 to 5 hetero atoms selected from the group consisting of N, O, S, Si and P,

or, taken together with the nitrogen atom to which they are attached, may form a saturated or unsaturated hydrocarbon-based heterocycle containing 3 to 10 and preferably 4 to 6 carbon atoms and optionally 1 to 5 other hetero atoms selected from the group consisting of N, O, S, Si and P, the ring optionally being substituted with at least one linear, branched or cyclic, saturated or unsaturated hydrocarbon-based radical containing 1 to 20, preferably 1 to 15 and better still 1 to 6 carbon atoms, optionally comprising 1 to 5 hetero atoms selected from the group consisting of N, O, S, Si and P;

R₄ may represent a linear, branched or cyclic, saturated or unsaturated hydrocarbon-based group containing 1 to 20, preferably 1 to 15 and better still 1 to 6 carbon atoms, optionally halogenated or perhalogenated, especially with F, Br and/or Cl, and/or optionally comprising 1 to 5 hetero atoms selected from the group consisting of N, O, S, Si and P.

In the present instance, R₅ and R₆ may represent, independently of each other, a group selected from the group consisting of:

the saturated cyclic aminoaryl groups of formula (IIA) or (IIB):

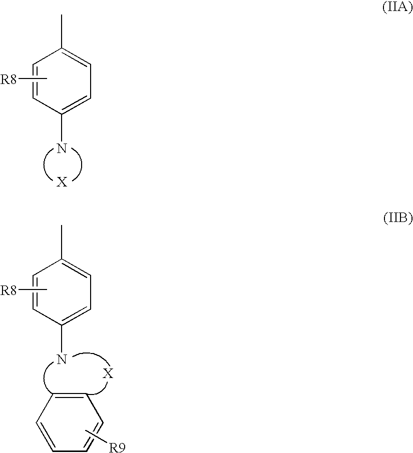

in which the ring comprising N and X is a saturated ring which contains in total 3 to 30, preferably 4 to 10 and better still 5, 6 or 7 atoms, including nitrogen, the rest being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20, preferably 1 to 15 and better still 1 to 6 carbon atoms, optionally comprising 1 to 5 hetero atoms selected from the group consisting of N, O, S, Si and P;

a linear, branched or cyclic, saturated or unsaturated hydrocarbon-based group containing 1 to 30, preferably 2 to 18 and better still 3 to 12 carbon atoms, optionally comprising 1 to 5 hetero atoms selected from the group consisting of N, O, S, Si and P; and especially a group selected from the group consisting of —C₆H₄—CONR₂R₃, —C₆H₄—NR₂R₃ and —C₆H₄—OR₄ with R₂, R₃ and R₄ having the meanings given above.

In particular, R₇ may represent a group selected from the group consisting of:

(i) linear, branched or cyclic, saturated or unsaturated hydrocarbon-based groups containing 1 to 30, preferably 1 to 18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;

(ii) halogen atoms, especially F, Br and/or Cl;

(iii) —CN (nitrile), —COOH (carboxylate), —NO₂ (nitro), —N═N— (azo), ═NH (imino) or —CONH₂ (amide) groups;

(iv) a hydrogen atom;

(v) a group selected from the group consisting of —NR₂R₃, —OR₄ and —SR₄, with R₂, R₃ and R₄ having the meanings given above;

(vi) the radical R₇ also possibly forming, with one of the bonds “i”, “j”, “k” or “g, h” taken with the radical R₁, or “f” taken with the radical R₁, a saturated hydrocarbon-based ring containing in total 3 to 8, preferably 4 to 7 and better still 5 or 6 carbon atoms, optionally comprising 1 to 5 hetero atoms selected from the group consisting of N, O, S, Si and P.

In particular, R′₁ may represent hydrogen or a linear, branched or cyclic, saturated or unsaturated hydrocarbon-based group containing 1 to 30, preferably 1 to 18 and better still 1 to 12 carbon atoms, optionally comprising 1 to 5 hetero atoms selected from the group consisting of N, O, S, Si and P, and/or optionally halogenated or perhalogenated, especially with F, Br and/or Cl.

In particular, R′₂ represents hydrogen or a group selected from the group consisting of —NO₂, —NR₂R₃ and —C(O)NR₂R₃, in which R₂ and R₃ either represent, independently of each other, a linear, branched or cyclic, saturated or unsaturated hydrocarbon-based group containing 1 to 20, preferably 1 to 12 and better still 1 to 6 carbon atoms, optionally comprising 1 to 5 hetero atoms selected from the group consisting of N, O, S, Si and P; or, taken together with the nitrogen atom to which they are attached, form a saturated or unsaturated hydrocarbon-based heterocycle containing 3 to 10 and preferably 4 to 6 carbon atoms and optionally 1 to 5 other hetero atoms selected from the group consisting of N, O, S, Si and P, the ring optionally being substituted with at least one linear, branched or cyclic, saturated or unsaturated hydrocarbon-based radical containing 1 to 20, preferably 1 to 15 and better still 1 to 6 carbon atoms, optionally comprising 1 to 5 hetero atoms selected from the group consisting of N, O, S, Si and P.

Mention may be made more particularly of the organic dyes of formula (I) or (Ia) for which:

R₁ represents hydrogen; or a group —COOR with R being a saturated hydrocarbon-based radical containing 1 to 12 and preferably 1 to 6 carbon atoms, and especially a methyl or ethyl radical; or a group

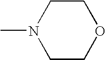

and/or

R₅ and R₆ represent, independently of each other, either (i) a group of formula (IIA):

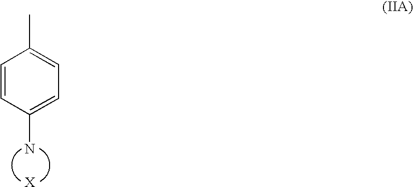

in which the ring comprising N and X is a saturated ring containing in total 4 to 7 atoms and especially 5 or 6 atoms, including nitrogen, and especially 3 to 5 carbon atoms and 0 or 1 oxygen atom; and in particular a group of formula:

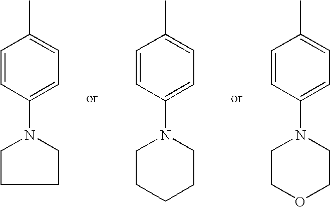

or (ii) a linear, branched or cyclic, saturated or unsaturated hydrocarbon-based group containing 5 to 14 and preferably 6 to 10 carbon atoms, optionally comprising 1 or 2 hetero atoms selected from the group consisting of N, O and S;

in particular a group

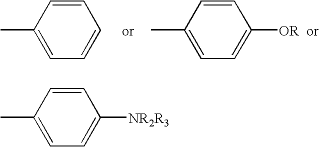

in which R is a saturated hydrocarbon-based radical containing 1 to 12 and preferably 1 to 6 carbon atoms, and especially a methyl or ethyl radical; and R₂ and R₃ are, independently of each other, a linear, branched or cyclic, saturated or unsaturated hydrocarbon-based group containing 1 to 20, preferably 1 to 15 and better still 1 to 6 carbon atoms, optionally comprising 1 to 5 hetero atoms selected from the group consisting of N, O, S, Si and P;

and/or

R₇ represents a hydrogen atom or a group —NR₂R₃, with R₂ and R₃ representing, independently of each other, a linear or branched, saturated hydrocarbon-based group containing 1 to 12 and preferably 1 to 6 carbon atoms, and especially a methyl and/or ethyl group.

Mention may also be made of the photochromic dyes of formula (II) or (IIa) for which:

R′₁ represents hydrogen or a group —COOR with R being a saturated hydrocarbon-based radical containing 1 to 12 and preferably 1 to 6 carbon atoms, and especially a methyl or ethyl radical;

and/or

R₅ and R₆ represent, independently of each other, either (i) a group of formula (IIA):

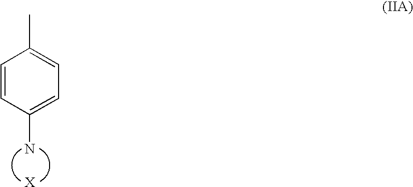

in which the ring comprising N and X is a saturated ring containing in total 4 to 7 atoms and especially 5 to 6 atoms, including nitrogen, and especially 4 to 5 carbon atoms and 0 or 1 oxygen atom; and in particular a group of formula:

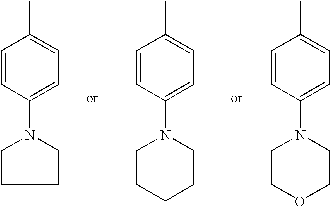

or (ii) a linear, branched or cyclic, saturated or unsaturated hydrocarbon-based group containing 5 to 14 and preferably 6 to 10 carbon atoms, optionally comprising 1 or 2 hetero atoms selected from the group consisting of N, O and S;

in particular a group:

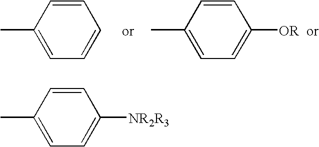

in which R is a saturated hydrocarbon-based radical containing 1 to 12 and preferably 1 to 6 carbon atoms, and especially a methyl or ethyl radical; and R₂ and R₃ are, independently of each other, a linear, branched or cyclic, saturated or unsaturated hydrocarbon-based group containing 1 to 20, preferably 1 to 15 and better still 1 to 6 carbon atoms, optionally comprising 1 to 5 hetero atoms selected from the group consisting of N, O, S, Si and P;

and/or

R′₂ represents hydrogen or a group —NR′R″, with R′ and R″, which may be identical or different, representing a linear or branched, saturated hydrocarbon-based group containing 1 to 12 and preferably 1 to 6 carbon atoms, and especially a methyl and/or ethyl group; or a group

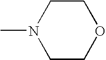

Non-limiting illustrations of dyes of naphthopyran type that may be mentioned include those described in patent applications WO 94/22850, WO 98/45281 and WO 00/18755.

More specifically, the following compounds are most particularly suitable for the invention:

3,3-di(4-methoxyphenyl)-6-morpholino-3H-naphtho[2, 1-b]pyran of formula:

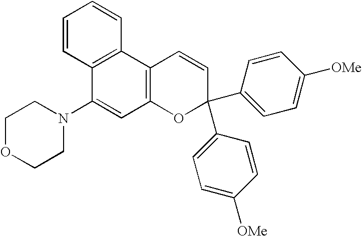

3-phenyl-3-(4-morpholinophenyl)-6-morpholino-3H-naphtho[2,1-b]pyran of formula:

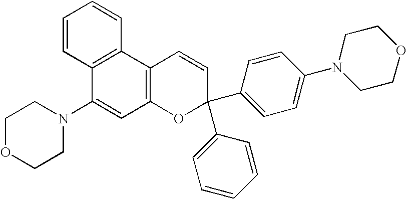

3-phenyl-3-(4-piperidinophenyl)-6-morpholino-3H-naphtho[2,1-b]pyran of formula:

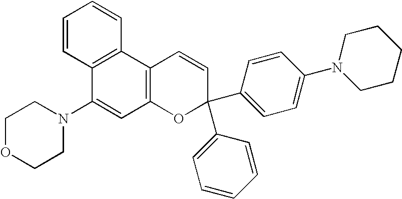

3-phenyl-3-(4-piperidinophenyl)-6-carboxymethyl-9-N-dimethyl-3H-naphtho[2,1-b]pyran of formula:

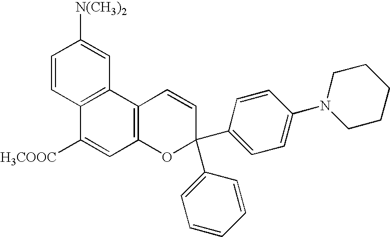

2-phenyl-2-(4-piperidinophenyl)-5-carboxymethyl-9-N-dimethyl-2H-naphtho[1,2-b]pyran of formula:

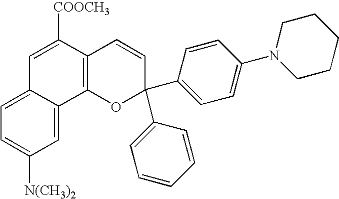

and mixtures thereof.

The naphthopyran derivative in accordance with the invention is present in a dissolved or dispersed form, more particularly dissolved in the composition according to the invention. Consequently, the choice of photochromic dyes that may be used in the context of the present invention is generally made taking into account their solubility in the phase or one of the phases of which the physiologically acceptable medium of the composition according to the invention is composed.

Other Dyes

For the purposes of the present invention, the expression “other dyes” or “second dye” is intended to cover dyes that are not photochromic in the sense of the present invention and preferably any compound selected from the group consisting of liposoluble and water-soluble monochromic dyes, nacres, reflective particles and goniochromatic coloring agents.

Goniochromatic Coloring Agents

For the purposes of the present invention, the term “goniochromatic coloring agent” denotes a coloring agent for obtaining, when the composition containing it is spread onto a support, a color path in the a*b* plane of the 1976 CIE (International Commission on Illumination) colorimetric space that corresponds to a variation Dh of the hue angle h of at least 20° when the angle of observation relative to the normal is varied between 0° and 80°, for an angle of light incidence of 45°.

The color path may be measured, for example, using an Instrument Systems Spectrogonioreflectometer of reference GON 360 Goniometer, after the cosmetic composition has been spread in fluid form to a thickness of 300 μm using an automatic spreader onto an Erichsen contrast card of reference Typ 24/5, the measurement being performed on the black background of the card.

The goniochromatic coloring agent may be present, for example, in an amount that can range, on a weight basis relative to the total weight of the composition, from 0.1% to 60%, or even from 1% to 20% or from 2% to 15%, and better still from 2% to 10% by weight, especially for a composition intended to be applied to the lips. A nail varnish composition may contain, for example, between 0.1% and 5% of goniochromatic coloring agent; a foundation may contain from 10% to 15% and a lipstick may contain from 2% to 8% by weight.

The goniochromatic coloring agent may be chosen so as to produce a relatively large color change with the angle of observation.

The goniochromatic coloring agent may be chosen, for example, from multilayer interference structures and liquid-crystal coloring agents.

In the case of a multilayer structure, it may comprise, for example, at least two layers, each layer, optionally independently of the other layer(s), being made, for example, from at least one material selected from the group consisting of the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys and polymers, and combinations thereof.

As regards the liquid-crystal goniochromatic particles that may be used in the composition according to the invention, they may especially be based on a polymer that may be obtained by polymerization of a monomer mixture comprising:

a) at least a first monomer A of formula (I) Y1-A1-M1-A2-Y2 in which

i) Y1 and Y2, which may be identical or different, represent a polymerizable group selected from the group consisting of acrylate or methacrylate groups, an epoxy group and an isocyanate, hydroxyl, vinyl ether (—O—CH═CH₂) or vinyl ester (—CO—O—CH═CH₂) group, and

ii) A1 and A2, which may be identical or different, represent a group of formula —CnH₂n—, in which n is an integer ranging from 0 to 20 and one or more methylene groups of the group —CnH₂n— possibly being replaced with one or more oxygen atoms, and

iii) M1 denotes a group of general formula (I′) —R₁—X₁—R₂—X₂—R₃—X₃—R₄—, in which R₁, R₂, R₃ and R₄, which may be identical or different, denote a divalent group selected from the group consisting of —O—, —COO—, —CONH—, —CO—, —S—, —C≡C—, —CH═CH—, —N═N—, —N═N(O)— and —R₂—X₂—R₃— or —R₂—X₂— or —R₂—X₂—R₃-X₃- also possibly being a single covalent bond, and X₁, X₂ and X₃ are identical or different groups selected from the group consisting of 1,4-phenylene and 1,4-cyclohexylene groups, the arylene or heteroarylene groups having an aryl nucleus containing from 6 to 10 atoms optionally substituted with B1 and/or B2 and/or B3, the heteroarylene containing from 1 to 3 hetero atoms selected from the group consisting of O, N and S atoms, cycloalkylene groups containing from 3 to carbon atoms optionally substituted with —B1 and/or —B2 and/or —B3, —B1, —B2, and —B3, which may be identical or different, being selected from the group consisting of C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups, and alkyl, alkoxy or alkylthio groups containing from 1 to 20 carbon atoms, interrupted with one or more oxygen atoms(s) or one or more sulphur atom(s) or one or more ester group(s),

and

b) at least a second monomer B, which is chiral, of formula (II) V1-A′1-W1-Z-W2-A′2-V2, in which

i) V1 and V2, which may be identical or different, denote a group selected from the group consisting of an acrylate or methacrylate group, an epoxy group, a vinyl ether or vinyl ester group, an isocyanate group, a C₁-C₂₀ alkyl, a C₁-C₂₀ alkoxy, a C₁-C₂₀ alkylthio, a (C₁-C₂₀)alkoxycarbonyl, a (C₁-C₂₀)alkylthiocarbonyl, —OH, —F, —Cl, —Br, —I, —CN, —NO₂, formyl and acetyl, and alkyl, alkoxy or alkylthio groups containing from 1 to 20 carbon atoms and interrupted with one or more oxygen atom(s) or one or more sulphur atom(s) or one or more ester (—CO—O—) group(s),

and at least V1 or V2 denotes a polymerizable group selected from the group consisting of acrylate or methacrylate groups, an epoxy group and an isocyanate, hydroxyl, vinyl ether (—O—CH═CH₂) or vinyl ester (—CO—O—CH═CH₂) group,

ii) A′1 and A′2, which may be identical or different, represent a group of formula —CₙH₂ₙ—, in which n is an integer ranging from 0 to 20 and one or more methylene groups of the group CₙH₂ₙ, possibly being replaced with one or more oxygen atoms, and

iii) W1 and W2 denote a divalent group of general formula R′₁—X′₁—R′₂—X′₂—R′₃— in which R′1, R′₂ and R′₃, which may be identical or different, denote a divalent group selected from the group consisting of —O—, —COO—, —CONH—, —CO—, —S—, —C≡C—, —CH═CH—, —N═N—, and —N═N(O)—, and R′₁, R′₂, R′₃ or R′₂—X′₂ may also be a single covalent bond, and X′₁ and X′₂ are identical or different groups selected from the group consisting of 1,4-phenylene and 1,4-cyclohexylene groups, the arylene or heteroarylene groups having an aryl nucleus containing from 6 to 10 atoms optionally substituted with B′1 and/or B′2 and/or B′3, the heteroarylene containing from 1 to 3 hetero atoms selected from the group consisting of O, N and S atoms, the cycloalkylene groups containing from 3 to 10 carbon atoms optionally substituted with —B′1 and/or —B′2 and/or —B′3, —B′1, -B′2, and —B′3, which may be identical or different, being selected from the group consisting of C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups, and alkyl, alkoxy or alkylthio groups containing from 1 to 20 carbon atoms, interrupted with one or more oxygen atoms(s) or one or more sulphur atom(s) or one or more ester group(s),

and Z denotes a divalent chiral group comprising at least 4 carbon atoms, especially from 4 to 20 carbon atoms and better still from 4 to 10 carbon atoms (the divalent chiral group comprising at least one asymmetric carbon, especially one or two asymmetric carbons and in particular two asymmetric carbons) and in particular a divalent chiral group derived from the family of dianhydrohexites, hexoses, pentoses, binaphthyl derivatives (binaphthyl groups), biphenyl derivatives (biphenyl groups), tartaric acid derivatives and optically active glycols.

The liquid-crystal polymer is preferably obtained by polymerization of a mixture of monomers comprising:

a) at least a first monomer A of formula (I) Y1-A1-M1-A2-Y2 in which

i) Y1 and Y2, which may be identical or different, represent an acrylate or methacrylate group, and preferably an acrylate group;

ii) A1 and A2, which may identical or different, represent a group of formula —CₙH₂n-, in which n is an integer ranging from 1 to 20, preferably from 2 to 6 and better still equal to 4;

iii) M1 denotes a group of general formula (I′) —R₁—X₁—R₂—X₂—R₃—X₃—R₄—, in which R₁ and R₄ denote —O—, and R₂ and R₃ denote —COO—,

and X₁, X₂ and X₃ are a 1,4-phenylene group, the carbonyl group —CO— of R₂ and of R₃, respectively, being linked to the group X₁ or X₃, respectively,

and

b) at least a second monomer B, which is chiral, of formula (II) V1-W1-Z-W2-V2, in which

i) V1 denotes an acrylate or methacrylate group, and preferably an acrylate group, and V2 denotes a C₁-C₂₀ alkyl, a C₁-C₂₀ alkoxy, a (C₁-C₂₀)alkoxycarbonyl or —OH group, and preferably denotes a C₁-C₂₀ and especially C₁-C₄ alkoxy group, and in particular a methoxy group;

ii) W1 represents a divalent group of formula —X′1-CO—O—,

W2 represents a divalent group of formula —O—CO—X′1-,

in which X′1 denotes a 1,4-phenylene group,

and Z denotes a chiral group containing two bonds, derived from the dianhydrohexite group, in particular a divalent radical of formula:

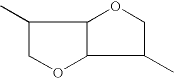

Preferably, the monomer mixture comprises from 70% to 99% by weight of monomer A and from 1% to 30% by weight of monomer B, and better still comprises from 90% to 95% by weight of monomer A and from 5% to 10% by weight of monomer B, relative to the total weight of monomer A and monomer B.

Preferably, the concentration of the polymerizable groups present in the mixture of monomer A and of monomer B (polymerizable groups Y1 and Y2 of monomer A and polymerizable groups V1 and V2 of monomer B) ranges from 3.2 to 15 mmol/g.

According to one particular embodiment of the invention, the liquid-crystal polymer is such that the mixture of monomer A and of monomer B comprises polymerizable groups, at least 90% of which are present in monomers containing at least two polymerizable groups, in a concentration ranging from 3.2 to 15 mmol/g.

In particular, the liquid-crystal polymer essentially comprises or consists of a mixture of the monomers A and B defined above.

The liquid-crystal polymer especially has a helix pitch of greater than 450 nm, especially ranging from 455 nm to 5 000 nm and in particular ranging from 455 nm to 1 000 nm, or even ranging from 455 nm to 650 nm.

The monomer A may have a weight-average molecular weight ranging from 150 to 800 and especially ranging from 460 to 625. The monomer A may be in particular an unsubstituted hydroquinone dibenzoate derivative.

The monomer B may have a weight-average molecular weight ranging from 500 to 1 000 and especially ranging from 500 to 700.

The liquid-crystal polymer may have a weight-average molecular weight of less than 625.

The liquid-crystal polymer defined above may be prepared according to the processes known in the prior art, such as those described in documents U.S. Pat. No. 5,362,315 and U.S. Pat. No. 5,807,497, starting with the monomer mixture described above.

The polymerization of the directed monomer mixture may be performed, in an already known manner, for example via a free-radical route with use of commercial thermal initiators, using electron beams or UV light in combination with commercial photoinitiators, or alternatively via addition reactions or condensation reactions.

The crosslinking of the monomer mixtures, in the chiral structural form, preferably takes place via a polyreaction which, depending on the type of polymerizable, polycondensable or polyadditionable groups, proceeds in the form of a free-radical, ionic or metal-catalysed polymerization, or a polycondensation reaction or a polyaddition reaction.

The initiation of the free-radical polymerization may be performed using corresponding initiators or via UV irradiation, using commercial photoinitiators or via high-energy irradiation, such as an electron beam. An advantage of thermal polymerization of the radicals or of polymerization via curing with electron beams lies in the fact that a light stabilizer, such as a UV (UVA) absorber or free-radical scavengers (HALS), may also be added to the polymerizable mixture to stabilize the pigments or the resulting films against UV light, for example for external applications, without resulting in losses in the polymerization conversion, as is the case during UV curing, due to the UVA-screening effect of the photoinitiator. There is thus no reduction in the crosslinking density.

If the curing of the films is performed with peroxide or with an electron beam, the monomer mixture preferably contains commercial light stabilizers, such as UV absorbers or free-radical scavengers, in an overall concentration of from 0.5% o 5% by weight.

Besides the photostabilizers, the monomer mixtures may also contain other common additives for inhibiting oxidation or inhibiting polymerization, or additives for improving the rheological properties. Furthermore, absorbent fillers, such as pigments or soot, and also fluorescent colorants or pigments, may be contained.

The film obtained after polymerization is then ground into particles, especially in the form of platelets.

Preferably, the particles of liquid-crystal polymer have a larger size ranging from 1 μm to 3 mm and preferably ranging from 30 μm to 500 μm. These particles are advantageously in the form of platelets.

The particles may be separated (sorted) via a process with grain size selectivity.

Such polymers and their particles are described in patent application EP-A-1 046 692.

Liquid-crystal polymer particles that may especially be used include those known under the CTFA name Polyacrylate-4 and sold under the names “Helicone® HC Sapphire”, “Helicone® HC Scarabeus”, “Helicone® HC Jade”, “Helicone® HC Maple”, “Helicone® HC XL Sapphire”, “Helicone® HC XL Scarabeus”, “Helicone® HC XL Jade” and “Helicone® HC XL Maple” by the company Wacker.

The particles of the liquid-crystal polymer may be present in the composition according to the invention in a content ranging from 0.01% to 99% by weight, especially ranging from 0.1% to 60% by weight, in particular ranging from 1% to 30% by weight and even ranging from 5% to 15% by weight, relative to the total weight of the composition.

Liposoluble and Water-Soluble Monochromatic Dyes

The composition may comprise, as second dye, at least one monochromatic dye, especially a natural organic dye such as cochineal carmine, and/or a synthetic dye such as halo acid dyes, azo dyes or anthraquinone dyes. Mention may also be made of mineral dyes such as copper sulphate or iron sulphate. Mention may also be made of Sudan brown, Sudan red and annatto, and also beetroot juice, carotene and methylene blue.

The dye may be present in the composition alone or as a mixture, for example in a proportion of from 0.001% to 15% by weight, especially from 0.01% to 5% by weight and in particular from 0.1% to 2% by weight, relative to the total weight of the composition.

Pigments

The term “pigments” should be understood as meaning white or colored, mineral or organic particles intended to color and/or opacify the composition.

Pigments may be present in the composition, as second dye, for example in a proportion of from 0.01% to 25% by weight and preferably in a proportion from 3% to 10% by weight of the final composition.

They may be white or colored, mineral or organic, and of standard or nanometric size. They may be in the form of powder or of pigmentary paste. Mention may be made of titanium oxide, zirconium oxide or cerium oxide, and also zinc oxide, iron oxide, or chromium oxide, ferric blue, chromium hydrate, carbon black, ultramarines (aluminosilicate polysulphides), manganese pyrophosphate and certain metal powders such as silver or aluminium powder. Mention may also be made of the D&C pigments and lakes commonly used to give the lips and the skin a makeup effect, which are calcium, barium, aluminium, strontium or zirconium salts.

Nacres

The term “nacres” should be understood as meaning iridescent particles that reflect light, which are produced especially by certain molluscs in their shell, or alternatively which are synthesized.

Nacres may be present in the composition, as second dye, for example in a proportion of from 0.01% to 20% by weight and preferably in a content of about from 3% to 10% by weight. Among the nacres that may be envisaged, mention may be made of natural nacre, mica coated with titanium oxide, with iron oxide, with natural pigment or with bismuth oxychloride, and also colored titanium mica.

Reflective Particles

For the purposes of the present invention, the term “reflective particles” denotes particles for which the size, the structure, in particular the thickness of the layer(s) of which they are made and the physical and chemical natures thereof, and the surface state, allow them to reflect incident light with a sufficient intensity to be able to create at the surface of the claimed composition, when this composition is applied to the support to be made up, highlight points that are visible to the naked eye, i.e. brighter points that contrast with their environment and appear to shine.

Reflective particles may be present in the composition, by way of second dye, by being uniformly dispersed, for example in a content ranging from 0.1% to 20%, especially from 1% to 15% by weight and in particular from 1% to 10% by weight, for example about 2%, relative to the total weight of the composition, especially for a composition intended to be applied to the lips.

Reflective particles that allow a metallic reflection of the incident light are most particularly suitable. This is the case especially when the reflective particles, irrespective of their shape, allow reflection on a layer of a metal, for example of silver. Such particles are found to be relatively neutral with respect to the color of the composition.

Reflective particles that may be used in the invention, with a metallic or white glint, can, for example, reflect light in all the components of the visible region without significantly absorbing one or more wavelengths. The spectral reflectance of these reflective particles may be, for example, greater than 70% in the 400-700 nm range, and in particular at least 80%, or even 90% or 95%.

The light reflected by the reflective particles may be non-iridescent, especially in the case of a metallic glint.

The reflective particles, irrespective of their shape, may or may not have a multilayer structure, and, in the case of a multilayer structure, for example at least one layer of uniform thickness, especially of a reflective material.

The reflective material may include a layer of metal or of a metallic compound.

Glass particles coated with a metallic layer are described especially in documents JP-A-09 188 830, JP-A-10 158 450, JP-A-10 158 541, JP-A-07 258 460 and JP-A-05 017 710.

The composition may also comprise, as second dye, at least one compound that is photochromic but different from those under consideration according to the invention, i.e. characterized generally by a ΔE value <5 and, where appropriate, insolubility in the composition according to the invention. Mention may be made especially of mineral photochromic compounds and more particularly doped aluminosilicates such as halogen-doped sodalite, or metal oxides or hydrates such as titanium oxides rendered photochromic with the aid of a metal selected from the group consisting of iron, chromium, copper, nickel, manganese, cobalt or molybdenum, in elemental form or in the form of a salt such as a sulphate, a chlorate, a nitrate or an acetate. Such a photochromic compound may be incorporated into the composition in an amount of from 0.001% to 20% by weight and preferably in an amount of from 0.1% to 10% by weight relative to the total weight of the composition.

Physiologically Acceptable Medium

The physiologically acceptable medium will be adapted to the nature of the surface to be made up or treated onto which the composition is to be applied, and also to the form in which the composition is intended to be packaged, especially solid or fluid at room temperature and atmospheric pressure.

The composition according to the invention may comprise at least one aqueous phase and/or at least one fatty phase.

The composition according to the invention may be anhydrous, or may also comprise an aqueous phase, which may comprise water, a floral water such as cornflower water and/or a mineral water such as eau de Vittel, eau de Lucas or eau de La Roche Posay.

The aqueous phase may comprise from 0.1% to 14% by weight, relative to the total weight of the aqueous phase, of a C₂-C₆ monoalcohol, for instance ethanol, propanol, butanol, isopropanol or isobutanol.

Oils

Generally, the composition according to the invention comprise(s) at least one oily phase, which may comprise one or more cosmetically acceptable oils.

According to the invention, the term “cosmetically acceptable oil” means any fatty substance that is liquid at 25° C. and 1 atm, with a molecular weight of greater than or equal to 160, especially between 170 and 106, or even between 200 and 5 105, which is compatible with application to the skin, mucous membranes (the lips) and/or the integuments (nails, eyelashes, eyebrows or hair).

Preferably, the oily phase is macroscopically homogeneous, i.e. homogeneous to the naked eye.

The oily phase may comprise one or more oils, which may be polar or apolar, volatile or non-volatile and, especially, hydrocarbon-based.

The term “polar oil” means an oil composed of chemical compounds comprising at least one polar group. Polar groups are well known to those skilled in the art: they may especially be groups of alcohol, ester or carboxylic acid type.

In particular, the polar oils according to the invention may be defined as having a mean solubility parameter δₐ according to the Hansen solubility space, at 25° C., of: δₐ≧5.0 (J/cm³)½.

The polar oils comprise the relatively polar oils that have a mean solubility parameter at 25° C. of: 5.0≦δₐ≦7.0 (J/cm³)½, and the definitely polar oils, which have a mean solubility parameter at 25° C. of: δₐ>7.0 (J/cm³)½.

Similarly, for the purposes of the invention, the apolar oils have a mean solubility parameter δₐ according to the Hansen solubility space, at 25° C., of: 0≦δₐ<5.0 (J/cm³)½.

For the purposes of the invention, the apolar oils comprise the definitely apolar oils (δₐ=0) and the sparingly polar oils, which have a mean solubility parameter at 25° C. of: 0<δₐ<5.0 (J/cm³)2.

Thus, the higher the value of δₐ, the higher the polarity of the oil.

The definition and calculation of the solubility parameters in the three-dimensional Hansen solubility space are described in the article by C. M. Hansen: “The three dimensional solubility parameters”, J. Paint Technol. 39, 105 (1967).

According to this Hansen space:

δ_{D} characterizes the London dispersion forces derived from the formation of dipoles induced during molecular impacts;

δₚ characterizes the Debye interaction forces between permanent dipoles and also the Keesom interaction forces between induced dipoles and permanent dipoles;

δₕ characterizes the specific interaction forces (such as hydrogen bonding, acid/base, donor/acceptor, etc.); δₐ is determined by the equation: δₐ=(δₚ²+δₕ²)½

The parameters δₚ, δₕ, δ_{D} and δₐ are expressed in (J/cm³)½.

When the oily phase is a mixture of different oils, the solubility parameters of the mixture are determined from those of the compounds taken separately, according to the following relationships:

$${δ}_{D m e l} = \frac{Σ}{i} x i δ D i ; δ = \frac{Σ}{i} x i δ p i and δ = \frac{Σ}{i} x i δ h i$$

$${δ}_{amel} = ( {δ}_{pmel}^{2} + {δ}_{hmel}^{2} ) 1 / 2$$

in which xi represents the volume fraction of the compound i in the mixture.

It is within the capability of a person skilled in the art to determine the amounts of each oil in order to obtain an oily phase that satisfies the desired criteria.

According to a first embodiment of the invention, the oily phase is polar and may mainly, or even exclusively, comprise one or more polar oils (relatively or definitely polar) as a mixture, which may thus represent 5% to 100% by weight, especially from 10% to 90% or even from 15% to 60%, and in particular from 20% to 50% by weight, relative to the total weight of the oily phase.

According to this embodiment, the polar oily phase has a mean solubility parameter δₐ according to the Hansen solubility space, at 25° C., of greater than or equal to 5.0 (J/cm³)½, especially greater than or equal to 5.3 or even greater than or equal to 5.5, and better still greater than or equal to 6.0 (J/cm³)½ or even greater than or equal to 7.0 (J/cm³)½.

According to a second particular embodiment of the invention, the oily phase is apolar and may comprise 5% to 100% by weight, especially 10 to 90%, or even 15 to 60% and better still 20 to 50% by weight of one or more apolar (apolar or sparingly polar) oils; it has a mean solubility parameter 8a according to the Hansen solubility space, at 25° C., of less than 5.0, especially less than or equal to 4.9, better still less than or equal to 4.5 and even better still less than or equal to 4.0 (J/cm³)½.

The oils that may be used in the oily phase may be chosen, alone or as a mixture, from volatile or non-volatile oils of animal, plant, mineral or synthetic origin.

The term “volatile oils” means oils that have, at 25° C., a vapour pressure of between 0.02 and 300 mmHg (i.e. 2.66 to 40 000 Pa). Preferably, volatile oils with a flash point of about 30-100° C. are used.

Mention may be made especially of:

animal or plant oils, especially formed from fatty acid esters of polyols, in particular liquid triglycerides, for example sunflower oil, maize oil, soybean oil, marrow oil, grapeseed oil, sesame seed oil, hazelnut oil, apricot oil, almond oil or avocado oil; fish oils or glyceryl tricaprocaprylate, or oils of plant or animal origin of formula R₁COOR₂ in which R₁ represents a higher fatty acid residue containing from 7 to 19 carbon atoms and R₂ represents a branched hydrocarbon-based chain containing from 3 to 20 carbon atoms, for example Purcellin oil; liquid paraffin, liquid petroleum jelly, beauty-leaf oil, macadamia oil, rapeseed oil, coconut oil, groundnut oil, palm oil, castor oil, jojoba oil, olive oil or cereal germ oil; shea butter oil; perhydrosqualene;

synthetic esters and ethers, especially of fatty acids, for instance the oils of formula R₁COOR₂ in which R₁ represents a higher fatty acid residue containing from 7 to 29 carbon atoms and R₂ represents a hydrocarbon-based chain containing from 3 to 30 carbon atoms, for instance isopropyl myristate, 2-ethylhexyl palmitate, 2-octyldodecyl stearate, 2-octyldodecyl erucate, isononyl isononanoate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate or diethylene glycol diisononanoate; pentaerythritol esters, for instance pentaerythrityl tetraisostearate; esters of the type such as tridecyl trimellitate;

fatty alcohols containing from 12 to 26 carbon atoms, for instance octyldodecanol, 2-butyloctanol, 2-hexyldecanol, 2-undecylpentadecanol or oleyl alcohol;

linear or branched hydrocarbons of mineral or synthetic origin, such as liquid paraffins and derivatives thereof, petroleum jelly, polydecenes, and hydrogenated polyisobutene such as parleam; isoparaffins, for instance isohexadecane and isodecane;

glycerides and especially acetylglycerides or triglycerides of fatty acids containing 4 to 10 carbon atoms, for instance heptanoic, octanoic and capric/caprylic acid triglycerides,

and mixtures thereof.

Among the polar oils that are particularly preferred, mention may be made of octyldodecanol, hexyldecanol, octyldecanol, oleyl alcohol, castor oil, diisostearyl malate, glyceryl triheptanoate, glyceryl trioctanoate, capric/caprylic acid triglyceride, triisononanoin and tridecyl trimellitate, and mixtures thereof.

Among the apolar oils that are particularly preferred, mention may be made of aliphatic hydrocarbons, especially of C₆-C₄₀, for instance volatile liquid paraffins, such as isohexadecane or isododecane, or non-volatile liquid paraffins, and derivatives thereof; petroleum jelly, hydrogenated or non-hydrogenated polydecenes, hydrogenated polyisobutene such as parleam oil, squalane, polybutylenes and isononyl isononanoate; fluoro oils and especially perfluoro oils, and mixtures thereof.

Mention may be made especially of the following oils:

| | δₐ (J/cm³)^{1/2} |
|---|---|
| Castor oil | 9.09 |
| 2-Hexyldecanol | 8.55 |
| Oleyl alcohol | 8.17 |
| Octyldodecanol | 7.69 |
| Heptanoic acid triglyceride | 7.29 |
| Diisostearyl malate | 7.19 |
| Octanoic acid triglyceride | 6.87 |
| Capric/caprylic acid triglyceride | 6.69 |
| Triisononanoin | 6.54 |
| Tridecyl trimellitate | 5.35 |
| Isononyl isononanoate | 4.87 |
| Hydrogenated polyisobutene | 0 |
| Isododecane | 0 |

The oily phase is preferably chosen such that the photochromic dye(s) are, if not soluble, then at least dispersible as they are, i.e. without an associated surface treatment.

The oily phase is preferably included in an amount of from 1% to 99% by weight, especially 10% to 90% by weight and preferably 15% to 80% by weight, relative to the total weight of the cosmetic composition.

The composition according to the invention may also comprise fatty substances other than the above oils, which may be chosen by a person skilled in the art on the basis of his general knowledge, so as to give the final composition the desired properties, for example in terms of consistency and/or texture.

These additional fatty substances may be waxes, gums and/or pasty fatty substances of animal, plant, mineral or synthetic origin, and also mixtures thereof.

For the purposes of the present invention, a wax is a lipophilic fatty compound, which is solid at room temperature (25° C.), with a reversible solid/liquid change of state, which has a melting point of greater than 40° C. and which may be up to 200° C., which generally has a hardness of greater than 0.5 MPa and which has an anisotropic crystal organization in the solid state.

Mention may be made especially of waxes of animal, plant, mineral or synthetic origin, such as microcrystalline waxes, paraffin wax, petrolatum, petroleum jelly, ozokerite or montan wax; beeswax, lanolin wax and derivatives thereof; candelilla wax, ouricury wax, carnauba wax, Japan wax, cocoa butter, cork fibre wax or sugar cane wax, lignite wax, rice bran wax, fir tree wax and cotton wax; hydrogenated oils with a melting point of greater than (about) 40° C., for instance hydrogenated jojoba oil; fatty esters and glycerides that are solid at 25° C.; polyethylene waxes and the waxes obtained by Fischer-Tropsch synthesis; hydrogenated oils that are solid at 25° C.; lanolins.

The pasty fatty substances generally have a melting point of between 25 and 60° C. and preferably between 30 and 45° C., and/or a hardness ranging from 0.001 to 0.5 MPa and preferably between 0.005 and 0.4 MPa. Mention may be made especially of lanolins and derivatives thereof, or cholesterol esters.

These additional fatty substances may be present in an amount of 0.1 to 50% by weight, especially 3 to 40% by weight and better still 5 to 30% by weight, relative to the total weight of the composition.

Surfactants

The composition according to the invention may optionally comprise one or more surfactants, especially when it is in the form of an emulsion, in particular in an amount of from 0.01% to 30% by weight relative to the total weight of the composition.

Mention may be made, alone or as a mixture, of alkali metal salts, ammonium salts, amine salts or amino alcohol salts of the following compounds: alkyl sulphates, alkyl ether sulphates, alkylamide sulphates and ether sulphates, alkylarylpolyether sulphates, monoglyceride sulphates, alkyl sulphonates, alkylamide sulphonates, alkylarylsulphonates, α-olefin sulphonates, paraffin sulphonates, alkyl sulphosuccinates, alkyl ether sulphosuccinates, alkylamide sulphosuccinates, alkyl sulphosuccinamates, alkyl sulphoacetates, alkylpolyglycerol carboxylates, alkyl phosphates/alkyl ether phosphates, acyl sarcosinates, alkyl polypeptidates, alkylamidopolypeptidates, acyl isethionates, alkyl laurates. The alkyl or acyl radical of all these compounds generally denotes a chain of 12 to 18 carbon atoms.

Mention may also be made of soaps and salts of fatty acids such as oleic acid, ricinoleic acid, palmitic acid, stearic acid and the acids of coconut oil or of hydrogenated coconut oil, and especially amine salts such as amine stearates; acyl lactylates in which the acyl radical contains 8-20 carbon atoms; polyglycol ether carboxylic acids.

Mention may also be made of polyethoxylated, polypropoxylated or polyglycerolated fatty alcohols, fatty alkylphenols and fatty acids, with a fatty chain containing from 8 to 18 carbon atoms; copolymers of ethylene oxide and of propylene oxide, condensates of ethylene oxide and of propylene oxide on fatty alcohols, polyethoxylated fatty amides, polyethoxylated fatty amines, ethanolamides, fatty acid esters of glycol, oxyethylenated or non-oxyethylenated fatty acid esters (stearate or oleate) of sorbitan, fatty acid esters of sucrose, fatty acid esters of polyethylene glycol (polyethylene glycol monostearate or monolaurate); phosphoric triesters, fatty acid esters of glucose derivatives; alkylpolyglycosides and alkylamides of amino sugars; the products of condensation of an α-diol, of a monoalcohol, of an alkylphenol, of an amide or of a diglycolamide with glycidol or a glycidol precursor.

Mention may also be made of trioleyl phosphate; fatty acid esters of polyols, for instance glyceryl or sorbitol mono-, di-, tri- or sesquioleates or stearates, and glyceryl or polyethylene glycol laurates; alkyl or alkoxy dimethicone copolyols with an alkyl or alkoxy chain that is pendent or at the end of the silicone skeleton, for example containing from 6 to 22 carbon atoms; polyoxyethylenated alkyl (lauryl, cetyl, stearyl or octyl) ethers and dimethicone copolyols.

Thickener

The composition according to the invention may also comprise one or more thickeners, for example in concentrations of from 0.01% to 6% by weight relative to the total weight of the composition.

The thickener may be chosen, alone or as a mixture, from:

polysaccharide biopolymers, for instance xanthan gum, carob gum, guar gum, alginates, modified celluloses such as hydroxyethylcellulose, methylcellulose, hydroxypropylcellulose and carboxymethylcellulose, starch derivatives, cellulose ether derivatives containing quaternary ammonium groups, and cationic polysaccharides;

synthetic polymers, for instance polyacrylic acids such as polyglyceryl (meth)acrylate polymers such as Hispagel or Lubragel from the companies Hispano Quimica or Gardian, polyvinylpyrrolidone, polyvinyl alcohol, crosslinked polymers of acrylamide and of ammonium acrylate, such as PAS 5161 or Bozepol C from Hoechst; acrylate/octylacrylamide copolymers, such as Dermacryl from National Starch; polyacrylamide-based polymers, such as Sepigel 305 from SEPPIC, crosslinked polymers of acrylamide and of methacryloyloxyethyltrimethylammonium chloride, such as Salcare SC 92 from Allied Colloids,

magnesium aluminium silicate;

optionally modified clays, for instance hectorites modified with a C₁₀ to C₂₂ fatty acid ammonium chloride, for instance hectorite modified with distearyldimethyl-ammonium chloride;

optionally modified silica;

galactomannans comprising one to six and better still from two to four hydroxyl groups per saccharide, substituted with a saturated or unsaturated alkyl chain, for instance guar gum alkylated with C₁ to C₆ and better still C₁ to C₃ alkyl chains and more particularly ethylated guar with a degree of substitution of 2 to 3, such as the product sold by the company Aqualon under the name N-Hance-AG;

cellulose derivatives such as ethylcellulose;

block copolymers, especially of “diblock” or “triblock” type, such as polystyrene/polyisoprene, polystyrene/polybutadiene, polystyrene/copoly(ethylene-butylene) or polystyrene/copoly(ethylene-propylene) such as those sold under the name “Kraton” by Shell Chemical;

polymers of polyamide type, for example comprising a polymer skeleton containing amide repeating units, and optionally at least one pendent fatty chain and/or at least one terminal chain, which are optionally functionalized, containing from 8 to 120 carbon atoms and being linked to these amide units, among which mention may be made of the products sold by the company Arizona Chemical under the names Uniclear 80 and Uniclear 100, which are a mixture of copolymers of a C₃₆ diacid condensed with ethylenediamine, with a weight-average molecular mass of about 6 000, and terminal ester groups resulting from the esterification of the remaining acid end groups with cetyl alcohol or stearyl alcohol, or mixtures thereof (also known as cetylstearyl alcohol).

Film-Forming Polymer

Depending on the intended application, the composition may also comprise at least one film-forming polymer. This is generally the case when it is desired to prepare a composition such as a nail varnish, a mascara, an eyeliner or a lip gloss.

Among the film-forming polymers that may be used in the composition of the present invention, mention may be made of synthetic polymers, of free-radical type or of polycondensate type, and polymers of natural origin, and mixtures thereof.

The film-forming polymers of free-radical type may especially be vinyl polymers or copolymers, especially acrylic polymers.

The vinyl film-forming polymers may result from the polymerization of ethylenically unsaturated monomers containing at least one acid group and/or esters of these acidic monomers and/or amides of these acidic monomers, for instance α,β-ethylenic unsaturated carboxylic acids such as acrylic acid, methacrylic acid, crotonic acid, maleic acid or itaconic acid.

The vinyl film-forming polymers may also result from the homopolymerization or copolymerization of monomers selected from the group consisting of vinyl esters, for instance vinyl acetate, vinyl neodecanoate, vinyl pivalate, vinyl benzoate and vinyl t-butylbenzoate and styrene monomers, for instance styrene and α-methylstyrene. In particular, these monomers may be polymerized with acidic monomers and/or esters thereof and/or amides thereof, such as those mentioned above.

Among the film-forming polycondensates that may be mentioned are polyurethanes, polyesters, polyesteramides, polyamides, epoxy ester resins and polyureas.

The polymers of natural origin, which are optionally modified, may be selected from the group consisting of shellac resin, sandarac gum, dammar resins, elemi gums, copal resins, water-insoluble cellulose-based polymers such as nitrocellulose, cellulose acetate, cellulose acetobutyrate, cellulose acetopropionate or ethylcellulose, and mixtures thereof.

The polymers may be dissolved or dispersed in the cosmetically acceptable medium. The polymers may be present in a content ranging from 0.01% to 40% by weight relative to the total weight of the composition.

The composition may also comprise a plasticizer selected from the group consisting of the usual plasticizers, which may be present in a content ranging from 0.1% to 40% by weight relative to the total weight of the composition.

Filler

The composition may also comprise fillers usually used in cosmetic compositions.

The term “fillers” should be understood as meaning colorless or white, mineral or synthetic, lamellar or non-lamellar particles intended to give the composition body or rigidity, and/or give the makeup softness, a matt effect and uniformity.

The fillers, which may be present in a proportion for example of from 0.01% to 60% by weight and preferably 3% to 10%, in the composition, may be mineral or synthetic, and lamellar or non-lamellar. Mention may be made of talc, mica, silica, kaolin, Nylon powder, polyethylene powder, Teflon, starch, boron nitride, polymer microspheres such as Expancel (Nobel Industrie), Polytrap (Dow Corning) and silicone resin microbeads (for example Tospearls from Toshiba), precipitated calcium carbonate, magnesium carbonate, magnesium hydrocarbonate and metal soaps derived from organic carboxylic acids containing from 8 to 22 carbon atoms.

Additives

The composition may also comprise a UV-screening agent, which may be incorporated into the composition in an amount of from 0.01% to 20% by weight and preferably in an amount of from 0.1% to 10% by weight relative to the total weight of the composition. Among the sunscreens that may be used, mention may be made especially of compounds belonging to the following families: para-aminobenzoics; salicylates; dibenzoylmethanes; cinnamates; β,β′,-diphenylacrylate derivatives; benzophenones; benzylidenecamphors; phenylbenzimidazoles; triazines; phenylbenzotriazoles; anthranilics; imidazolines and/or benzalmalonates.

The composition according to the invention may also be free of UV-screening agent.

The composition may also comprise any additive usually used in cosmetics, such as antioxidants, fragrances, essential oils, preserving agents, lipophilic or hydrophilic cosmetic active agents, moisturizers, vitamins, sphingolipids, self-tanning agents such as DHA, optical brighteners, antifoams or sequestering agents.

Needless to say, a person skilled in the art will take care to select the optional additional compounds, and/or the amount thereof, such that the advantageous properties of the composition according to the invention are not, or are not substantially, adversely affected by the envisaged addition.

Galenical Formulation

The cosmetic composition according to the invention especially finds a particularly advantageous application in the field of making up the skin, semi-mucous membranes, mucous membranes and/or the integuments (nails, eyelashes, eyebrows, body hair and head hair).

It may comprise or be inany form including the form of a suspension, a dispersion especially of oil in water by means of vesicles; an optionally thickened or even gelled oily solution; an oil-in-water, water-in-oil or multiple emulsion; a gel or a mousse; an oily or emulsified gel; a dispersion of vesicles, especially lipid vesicles; a two-phase or multi-phase lotion; a spray; a free, compact or cast powder; an anhydrous paste. This composition may have the appearance of a lotion, a cream, a salve, a soft paste, an ointment, a solid cast or moulded especially in stick or dish form, or a compacted solid.

The cosmetic composition according to the invention may be in the form of a care and/or makeup product for body or facial skin, the lips and the hair, an antisun or self-tanning product, or even a hair product.

It finds a particular application in the field of lipsticks, foundations, makeup rouges, eyeshadows, free or compact powders, tinted creams, body makeup products, skin-coloring products, eyeliners and mascaras.

The cosmetic composition according to the invention may be obtained according to the preparation processes conventionally used in cosmetics.

The examples given below are presented purely as illustrations and with no limitation on the field of the invention.

### EXAMPLES

The percentages below are all expressed on a weight basis relative to the total weight of the composition.

The photochromic dyes used in the formulations below are naphthopyran derivatives from the company James Robinson sold under the following names:

| | | |
|---|---|---|
| Reversacol | Corn Yellow | Ref. 1 |
| Reversacol | Flame | Ref. 2 |
| Reversacol | Aqua Green | Ref. 3 |
| Reversacol | Ruby | Ref. 4 |

Their respective ΔE values were assessed according to the protocol described in the description. Table I gives the differences in hue (ΔE) obtained after exposure for 2 minutes.

| Photochromic agents | ΔE |
|---|---|
| Ref. 1 | 51 |
| Ref. 2 | 53 |
| Ref. 3 | 6 |
| Ref. 4 | 49 |

To check the dynamic color effect properties of a composition in accordance with the invention, a foundation formulation having the composition below was prepared:

Foundation

| Composition | % |
|---|---|
| Water | qs 100 |
| Veegum | 0.7 |
| Propylene glycol | 6 |
| Polysorbate 20 | 3.7 |
| Iron oxides | 0.4 |
| Photochromic dyes: Ref. 1, 2 and 3 | 0.1 |
| Isononyl isononanoate | 22 |
| Stearic acid | 1 |
| Methyl glucose sesquistearate | 3.5 |
| Silicone | 10 |

The three photochromic dyes were simultaneously introduced therein: Ref. 1 in a proportion of 0.07% by weight, Ref 2 in a proportion of 0.02% by weight and Ref. 3 in a proportion of 0.01%.

The efficacy in terms of the color dynamics is checked for the corresponding photochromic composition. It thus suffices to incorporate a small concentration, especially of about 0.1% by weight, of photochromic dye(s) relative to the total weight of the composition in order for the cream, which is initially weakly tinted, to darken within a few seconds under exposure to sunlight.

Tube of lipstick

| | weight % |
|---|---|
| Octyldodecyl neopentanoate | 17.0 |
| Capric/caprylic acid triglyceride | 10.2 |
| Lanolin oil | 15.0 |
| Acetylated lanolin | 10.2 |
| Polybutene | 15.0 |
| Photochromic dye (Ref. 4) | 2.0 |
| Silver-coated glass particles (METASHINE ®)* | 2.0 |
| Goniochromatic pigment (SICOPEARL ®)** | 3.0 |
| Microcrystalline wax | 2.5 |
| Polyethylene wax | 7.4 |
| Phenyltrimethicone | 7.0 |
| Hydrogenated polyisobutene | 6.5 |
| Fragrance, preserving agent, antioxidant | qs |

*sold by the company TOYAL
**sold by the company BASF

A tube of lipstick that also has good color dynamic properties in response to an excitation with UV light is obtained.

The above written description of the invention provides a manner and process of making and using it such that any person skilled in this art is enabled to make and use the same, this enablement being provided in particular for the subject matter of the appended claims, which make up a part of the original description.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out. As used above, the term “selected from the group consisting of” includes mixtures of the listed materials.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A cosmetic composition comprising, in a physiologically acceptable medium, at least two dyes, including at least one photochromic dye, wherein the photochromic dye has a difference in hue ΔE at least equal to 5 and is at least one naphthopyran derivative of the 2H-naphtho[2,1-b]pyran type of formula (I) or the 3H-naphtho[2,1-b]pyran type of formula (II): 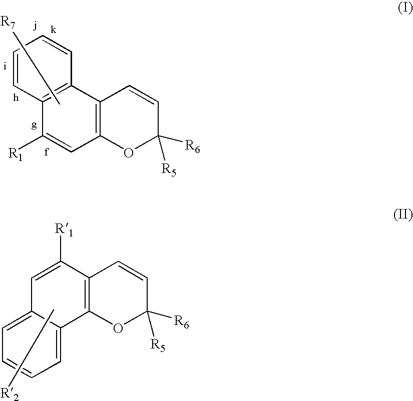
in which:
R₁ represents:
(i) a hydrogen atom;
(ii) a linear, branched or cyclic, saturated or unsaturated hydrocarbon-based group containing from 1 to 30 carbon atoms, optionally comprising 1 to 5 hetero atoms selected from the group consisting of N, O, S, Si and P, and/or optionally halogenated or perhalogenated;
(iii) a hydrocarbon-based ring formed with one of the bonds “f” or “gh” and the radical R₇; or
(iv) a group selected from the group consisting of —COOR₄, —C(O)NR₂R₃, —NR₂R₃, —OR₄ and —SR₄, in which:
R₂ and R₃ either represent, independently of each other, a linear, branched or cyclic, saturated or unsaturated hydrocarbon-based group containing 1 to 20 carbon atoms, optionally comprising 1 to 5 hetero atoms selected from the group consisting of N, O, S, Si and P,
or, taken together with the nitrogen atom to which they are attached, form a saturated or unsaturated hydrocarbon-based heterocycle containing 3 to 10 carbon atoms and optionally 1 to 5 other hetero atoms selected from the group consisting of N, O, S, Si and P, the ring optionally being substituted with at least one linear, branched or cyclic, saturated or unsaturated hydrocarbon-based radical containing 1 to 20 carbon atoms, optionally comprising 1 to 5 hetero atoms selected from the group consisting of N, O, S, Si and P;
R₄ represents a linear, branched or cyclic, saturated or unsaturated hydrocarbon-based group containing 1 to 20 carbon atoms, which is optionally halogenated or perhalogenated and/or optionally comprising 1 to 5 hetero atoms selected from the group consisting of N, O, S, Si and P;
R₅ and R₆ represent, independently of each other, a group selected from the group consisting of:
(i) the saturated cyclic aminoaryl groups of formula (IIA) or (IIB): 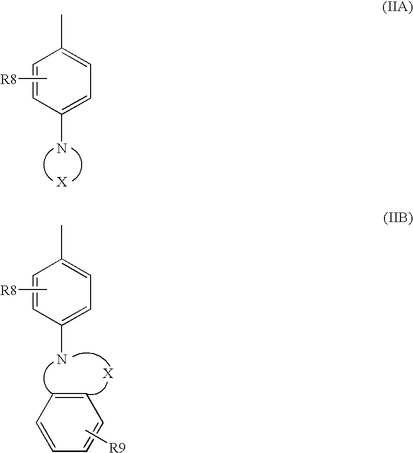
in which the ring comprising N and X is a saturated ring containing in total 3 to 30 atoms including the nitrogen, the remainder being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20 carbon atoms, optionally comprising 1 to 5 hetero atoms selected from the group consisting of N, O, S, Si and P;
(ii) the indolinoaryl groups of formula (III): 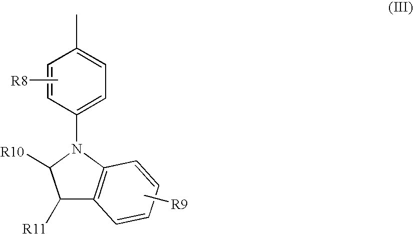
in which R₁₀ and R₁₁ represent, independently of each other, a group selected from the group consisting of (i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated; (ii) halogen atoms,; (iii) —CN (nitrile), —COOH (carboxylate) or —NO₂ (nitro) groups; (iv) a hydrogen atom; (v) a group selected from the group consisting of —C(O)NR₂R₃, —NR₂R₃, —OR₄ and —SR₄ with R₂, R₃ and R₄ having the meanings given above; (vi) the radicals R₁₀ and R₁₁ together possibly forming a saturated or unsaturated hydrocarbon-based ring containing in total 5 to 8 atoms (including the atoms of the indoline ring), the atoms being selected from the group consisting of C, O, S and/or NR with R representing H or a linear, branched or cyclic, saturated or unsaturated hydrocarbon-based radical containing 1 to 20 carbon atoms, optionally comprising 1 to 5 hetero atoms selected from the group consisting of N, O, S, Si and P,
(iii) the groups of formula (IV): 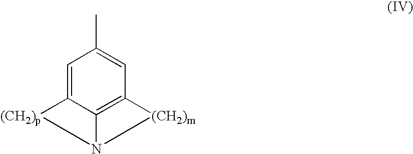
in which m and p are, independently of each other, integers ranging from 2 to 5;
(iv) the unsaturated cyclic aminoaryl groups of formula (VA), (VB) or (VC): 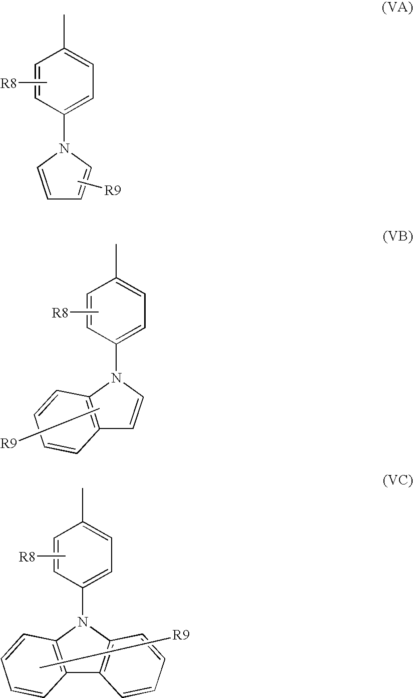
in which R₈ and R₉ represent, independently of each other, a group selected from the group consisting of (i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated; (ii) halogen atoms; (iii) —CN (nitrile), —COOH (carboxylate) or —NO₂ (nitro) groups; (iv) a hydrogen atom; (v) a group selected from the group consisting of —C(O)NR₂R₃, —NR₂R₃, —OR₄ and —SR₄ with R₂, R₃ and R₄ having the meanings given above;
(v) a linear, branched or cyclic, saturated or unsaturated hydrocarbon-based group containing 1 to 30 carbon atoms, optionally comprising 1 to 5 hetero atoms selected from the group consisting of N, O, S, Si and P; and especially a group selected from the group consisting of —C₆H₄—CONR₂R₃, —C₆H₄—NR₂R₃ and —C₆H₄—OR₄ with R₂, R₃ and R₄ having the meanings given above;
R₇ represents a group selected from the group consisting of:
(i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated;
(ii) halogen atoms;
(iii) —CN (nitrile), —COOH (carboxylate), —NO₂ (nitro), —N═N— (azo), ═NH (imino) or —CONH₂ (amide) groups;
(iv) a hydrogen atom;
(v) a group selected from the group consisting of —C(O)NR₂R₃, —NR₂R₃, —OR₄ and —SR₄ with R₂, R₃ and R₄ having the meanings given above;
(vi) the radical R₇ also possibly forming, with one of the bonds “i”, “j”, “k” or “g,h” taken with the radical R₁, or “f” taken with the radical R₁, a saturated hydrocarbon-based ring containing in total 3 to 8 carbon atoms, optionally comprising 1 to 5 hetero atoms selected from the group consisting of N, O, S, Si and P;
R′₁ represents a group selected from the group consisting of:
(i) a hydrogen atom;
(ii) a linear, branched or cyclic, saturated or unsaturated hydrocarbon-based group containing 1 to 30 carbon atoms, optionally comprising 1 to 5 hetero atoms selected from the group consisting of N, O, S, Si and P, and/or optionally halogenated or perhalogenated;
(iii) a group selected from the group consisting of —C(O)NR₂R₃, —NR₂R₃, —OR₄ and —SR₄, with R₂, R₃ and R₄ having the meanings given above;
R′₂ represents a group selected from the group consisting of:
(i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated;
(ii) halogen atoms;
(iii) —CN (nitrile), —COOH (carboxylate), —NO₂ (nitro), —N═N— (azo), ═NH (imino) or —CONH₂ (amide) groups;
(iv) a hydrogen atom;
(v) a group selected from the group consisting of —C(O)NR₂R₃, —NR₂R₃, —OR₄ and —SR₄, with R₂, R₃ and R₄ having the meanings given above.

2. The composition according to claim 1, wherein the photochromic dye is present in an amount that is effective to give the composition a dynamic effect in terms of color and radiance.

3. The composition according to claim 1, wherein the naphthopyran derivative is present in the composition in a dissolved form.

4. The composition according to claim 1, wherein the naphthopyran derivative corresponds to formula (Ia) or (IIa) below where variables R_{#} and R′_{#} are as defined in claim 1:

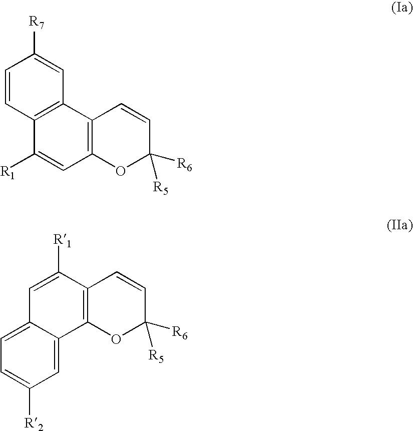

5. The composition according to claim 1, wherein R₁ represents a hydrogen atom; a hydrocarbon-based ring with one of the bonds “f” or “gh” and the radical R₇; or a group selected from the group consisting of —COOR₄, —NR₂R₃, —OR₄ and —SR₄, in which:
R₂ and R₃ either may represent, independently of each other, a linear, branched or cyclic, saturated or unsaturated hydrocarbon-based group containing 1 to 20, carbon atoms, optionally comprising 1 to 5 hetero atoms selected from the group consisting of N, O, S, Si and P,
or, taken together with the nitrogen atom to which they are attached, may form a saturated or unsaturated hydrocarbon-based heterocycle containing 3 to 10 carbon atoms and optionally 1 to 5 other hetero atoms selected from the group consisting of N, O, S, Si and P, the ring optionally being substituted with at least one linear, branched or cyclic, saturated or unsaturated hydrocarbon-based radical containing 1 to 20 carbon atoms, optionally comprising 1 to 5 hetero atoms selected from the group consisting of N, O, S, Si and P;
R₄ may represent a linear, branched or cyclic, saturated or unsaturated hydrocarbon-based group containing 1 to 20 carbon atoms, optionally halogenated or perhalogenated and/or optionally comprising 1 to 5 hetero atoms selected from the group consisting of N, O, S, Si and P;
and/or
R₅ and R₆ may represent, independently of each other, a group selected from the group consisting of:
the saturated cyclic aminoaryl groups of formula (IIA) or (IIB): 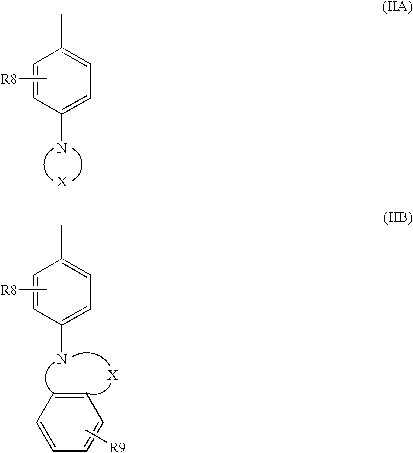
in which the ring comprising N and X is a saturated ring which contains in total 3 to 30 including nitrogen, the rest being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20 carbon atoms, optionally comprising 1 to 5 hetero atoms selected from the group consisting of N, O, S, Si and P;
a linear, branched or cyclic, saturated or unsaturated hydrocarbon-based group containing 1 to 30 carbon atoms, optionally comprising 1 to 5 hetero atoms selected from the group consisting of N, O, S, Si and P; and especially a group selected from the group consisting of —C₆H₄—CONR₂R₃, —C₆H₄—NR₂R₃ and —C₆H₄—OR₄ with R₂, R₃ and R₄ having the meanings given above.

6. The composition according to claim 1, wherein the photochromic dye corresponds to formula (I) for which:
R₁ represents hydrogen; or a group —COOR with R being a saturated hydrocarbon-based radical containing 1 to 12 carbon atoms, or a group 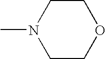
and/or
R₅ and R₆ represent, independently of each other, either (i) a group of formula (IIA): 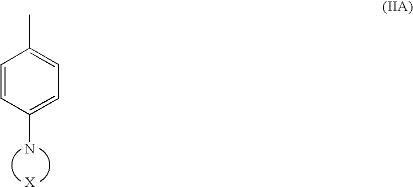
in which the ring comprising N and X is a saturated ring containing in total 4 to 7 atoms, including nitrogen, and especially 3 to 5 carbon atoms and 0 or 1 oxygen atom; and in particular a group of formula: 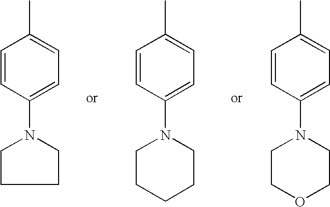
or (ii) a linear, branched or cyclic, saturated or unsaturated hydrocarbon-based group containing 5 to 14 carbon atoms, optionally comprising 1 or 2 hetero atoms selected from the group consisting of N, O or S;
and/or
R₇ represents a hydrogen atom or a group —NR₂R₃, with R₂ and R₃ representing, independently of each other, a linear or branched, saturated hydrocarbon-based group containing 1 to 12 carbon atoms.

7. The composition according to claim 1, wherein the photochromic dye corresponds to formula (II) for which:
R′₁ represents hydrogen or a group —COOR with R being a saturated hydrocarbon-based radical containing 1 to 12 carbon atoms;
and/or
R₅ and R6 represent, independently of each other, either (i) a group of formula (IIA): 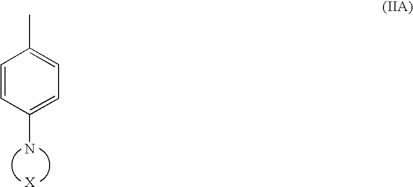
in which the ring comprising N and X is a saturated ring containing in total 4 to 7 atoms, including nitrogen,
or (ii) a linear, branched or cyclic, saturated or unsaturated hydrocarbon-based group containing 5 to 14 carbon atoms, optionally comprising 1 or 2 hetero atoms selected from the group consisting of N, O and S;
and/or
R′₂ represents hydrogen or a group —NR′R″, with R′ and R″, which may be identical or different, representing a linear or branched, saturated hydrocarbon-based group containing 1 to 12 carbon atoms.

8. The composition according to claim 1, wherein the photochromic dye is selected from the group consisting of:
3,3-di(4-methoxyphenyl)-6-morpholino-3H-naphtho[2,1-b]pyran
3-phenyl-3-(4-morpholinophenyl)-6-morpholino-3H-naphtho[2,1-b]pyran
3-phenyl-3-(4-piperidinophenyl)-6-morpholino-3H-naphtho[2,1-b]pyran
3-phenyl-3-(4-piperidinophenyl)-6-carboxymethyl-9-N-dimethyl-3H-naphtho[2,1 -b]pyran
2-phenyl-2-(4-piperidinophenyl)-5-carboxymethyl-9-N-dimethyl-2H-naphtho[1,2-b]pyran, and mixtures thereof.

9. The composition according to claim 1, comprising from 0.001% to 20% by weight of the photochromic dye, relative to the total weight of the cosmetic composition.

10. The composition according to claim 1, wherein the photochromic dye comprises at least two different naphthopyran derivatives.

11. The composition according to claim 1, combining with the photochromic dye at least a second dye.

12. The composition according to claim 11, wherein the second dye is selected from the group consisting of the group consisting of goniochromatic coloring agents, water-soluble or liposoluble monochromatic dyes, pigments, reflective particles and nacres, and mixtures thereof.

13. The composition according to claim 1, comprising, as a second dye, at least one goniochromatic coloring agent.

14. The composition according to claim 13, comprising from 0.1% to 60%, by weight of the goniochromatic coloring agent, relative to the total weight of the composition.

15. The composition according to claim 13, wherein the goniochromatic coloring agent is selected from the group consisting of multilayer interference structures and liquid-crystal coloring agents.

16. The composition according to claim 15, wherein the goniochromatic coloring agent of multilayer interference structure comprises at least two layers, each layer, optionally independently of the other layer(s), being made from at least one material selected from the group consisting of the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys and polymers, and combinations thereof.

17. The composition according to claim 15, comprising a liquid-crystal coloring agent obtained by polymerization of a monomer mixture comprising:
a) at least a first monomer A of formula (I) Y1-A1-M1-A2-Y2
in which
i) Y1 and Y2, which may be identical or different, represent an acrylate or methacrylate group;
ii) A1 and A2, which may be identical or different, represent a group of formula —CnH₂n-, in which n is an integer ranging from 1 to 20;
iii) M1 denotes a group of general formula (I′) —R₁—X₁—R₂—X₂—R₃—X₃—R₄—, in which R₁ and R₄ denote —O—, and R₂ and R₃ denote —COO—, and X₁, X₂ and X₃ are a 1,4-phenylene group, the carbonyl group —CO— of R₂ and of R₃, respectively, being linked to the group X₁ or X₃, respectively,
and
b) at least a second monomer B, which is chiral, of formula (II) V1-W1-Z-W2-V2, in which
i) Vi denotes an acrylate or methacrylate group, and V2 denotes a C₁-C₂₀ alkyl, C₁-C₂₀ alkoxy, (C₁-C₂₀)alkoxycarbonyl or —OH group,;
ii) W1 represents a divalent group of formula —X′1-CO—O—,
W2 represents a divalent group of formula —O—CO—X′1-,
in which X′1 denotes a 1,4-phenylene group,
and Z denotes a chiral group containing two bonds, derived from the dianhydrohexite group, in particular a divalent radical of formula:

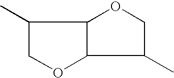

18. Composition according to claim 17, wherein the liquid-crystal coloring agent is obtained from a monomer mixture comprising from 70% to 99% by weight of monomer A and from 1% to 30% by weight of monomer B, relative to the total weight of monomer A and of monomer B.

19. The composition according to claim 15, comprising a liquid-crystal coloring agent present in a content ranging from 0.01% to 99% by weight, relative to the total weight of the composition.

20. The composition according to claim 1, comprising, as a second dye, from 0.001% to 15% by weight of at least one liposoluble or water-soluble dye relative to the total weight of the composition.

21. The composition according to claim 1, comprising, as a second dye, from 0.01% to 25% by weight of at least one pigment relative to the total weight of the composition.

22. The composition according to claim 1, comprising, as a second dye, from 0.01% to 20% by weight of nacres relative to the total weight of the composition.

23. The composition according to claim 1, comprising, as a second dye, from 0.1% to 20% of reflective particles relative to the total weight of the composition.

24. The composition according to claim 1, further comprising at least one oily phase.

25. The composition according to claim 24, wherein the oily phase comprises one or more polar or apolar, volatile or non-volatile oils.

26. The composition according to claim 24, wherein the oily phase comprises from 5% to 100% by weight, of polar oils relative to the total weight of the oily phase.

27. The composition according to claim 24, wherein the oily phase comprises from 5% to 100% by weight, of apolar oils relative to the total weight of the oily phase.

28. The composition according to claims 24, wherein the oils may be selected from the group consisting of volatile or non-volatile oils of animal, plant, mineral or synthetic origin, and mixtures thereof.

29. The composition according to claim 28, wherein the oils are selected from the group consisting of animal or plant oils, synthetic esters and ethers, especially of fatty acids, fatty alcohols, linear or branched hydrocarbons of mineral or synthetic origin, and glycerides, and mixtures thereof.

30. The composition according to claims 24, wherein the oily phase is such that the photochromic dye(s) is(are) soluble or dispersible therein.

31. The composition according to claims 24, wherein comprising from 1% to 99% by weight, of oily phase relative to the total weight of the composition.

32. The composition according to claim 1, comprising from 0.1% to 50% by weight, of a fatty substance other than an oil, relative to the total weight of the composition.

33. The composition according to claim 1, wherein said composition is anhydrous.

34. The composition according to claim 1, further comprising at least one aqueous phase.

35. The composition according to claim 34, wherein the aqueous phase comprises from 0.1% to 14% by weight of a C₂-C₆ monoalcohol, relative to the total weight of the aqueous phase.

36. The composition according to claim 1, further comprising at least one surfactant.

37. The composition according to claim 36, wherein said surfactant is present in an amount from 0.01% to 30% by weight relative to the total weight of the composition.

38. The composition according to claim 1, further comprising at least one thickener.

39. The composition according to claim 38, wherein said thickener is present in an amount from 0.01% to 6% by weight relative to the total weight of the composition.

40. The composition according to claim 1, further comprising at least one film-forming polymer.

41. The composition according to claim 40, wherein said film-forming polymer is present in an amount from 0.01% to 4% by weight relative to the total weight of the composition.

42. The composition according to claim 1, further comprising at least one filler.

43. The composition according to claim 42, wherein said filler is present in a proportion of from 0.01% to 60% by weight and in particular from 3% to 10% by weight relative to the total weight of the composition.

44. A process for making up the skin, the lips and/or the integuments, comprising applying the composition according to claim 1 to the skin, the lips and/or the integuments.

45. The process of claim 44, comprising applying said composition to the skin.

46. The process according to claim 45, wherein the photochromic dye is a naphthopyran.
